(19) United States
(12) Patent Application Publication
(10) Pub. No.: US 2008/0307541 A1
(43) Pub. Date: Dec. 11, 2008

(54) Transgenic plant expressing glutamyl-tRNA synthetase

(76) Inventor: Hsu-Liang Hsieh, Taipei (TW)

Correspondence Address: BIRCH STEWART KOLASCH & BIRCH, PO BOX 747, FALLS CHURCH, VA, 22040-0747, US

(21) Appl. No.: 11/384,385

(22) Filed: Mar. 21, 2006

(51) Int. Cl. C12N 15/82; C12N 15/00; A01H 5/00

(52) U.S. Cl. 800/278; 435/320.1; 800/298; 800/320; 800/320.1; 800/320.2; 800/320.3; 800/317.2; 800/317; 800/317.1; 800/310; 800/307; 800/315; 800/309; 800/316; 800/312; 800/317.4

(57) ABSTRACT

The present invention provides a DNA construct which comprises glutamyl-tRNA synthetase. Additionally, transgenic plants and tissues for the expression of glutamyl-tRNA synthesis are also provided. Furthermore, the present invention also provides methods for utilizing the DNA construct to produce the transgenic plants and tissues.

### BACKGROUND OF THE INVENTION

1. Field of the Invention

The present invention relates generally to a DNA construct and transgenic plants for the expression of glutamyl-tRNA synthetase.

2. Description of the Prior Art

Chlorophyll is the molecule that absorbs sunlight and uses its energy to synthesize carbohydrates from CO₂ and water. This process is known as photosynthesis and is the basis for sustaining the life processes of all plants. Since animals and humans obtain their food supply by eating plants, photosynthesis can be said to be the source of our life also. Through the photosynthesis process, plants can produce a large number of essential elements, such as glucose, sucrose, fructose, and cellulose. Furthermore, the condition of our skin and digestive system can be improved by obtaining chlorophyll-containing foods.

Research studies in humans have found that damage to DNA by aflatoxin can be decreased as much as 55% through supplementation with chlorophyllin, a derivative of chlorophyll. Studies has provided that chlorophyll has anti-inflammatory, antioxidant, and wound-healing properties, furthermore, it also can protects cells from exposure to carcinogens, mutagens and radiation.

Rice is a staple food for over half of the world's population. In parts of Africa and Asia, many poorer urban families get over half their daily calories from rice. As the world population increases, rice production has to be raised by at least 70% over the next three decades. If the contents of nutrition in rice can be enhanced, people may be healthier by just eating rice. A successful example of genetically engineered rice which has improved nutrition is the “Golden rice”. In 1999, Swiss and German scientists announced the development of a genetically engineered rice to produce β-carotene, a substance which the body can convert to Vitamin A. The new rice was quickly heralded as a miracle cure for vitamin A deficiency (VAD), a condition which afflicts millions of people in developing countries, especially children and pregnant women. Severe VAD can cause partial or total blindness; less severe deficiencies weaken the immune system, increasing the risk of infections such as measles and malaria. Women with VAD are more likely to die during or after childbirth. Each year, it is estimated that VAD causes blindness in 350,000 pre-school age children, and it is implicated in over one million deaths. Therefore, the development of the “Golden rice” is such important to save people from VAD.

The potato is the world's fourth most important food crop and by far the most important vegetable. Potatoes are currently grown commercially in nearly every state of the United States. Annual potato production exceeds 18 million tons in the United States and 300 million tons worldwide. The popularity of the potato derives mainly from its versatility and nutritional value. Potatoes can be used fresh, frozen or dried, or can be processed into flour, starch or alcohol. They contain complex carbohydrates and are rich in calcium, niacin and vitamin C. Furthermore, although potato chips and French fries in many reports tend to contain the most acrylamide, these food industrial products of potatoes still are very popular in the world.

According to the health benefit of chlorophyll and the popularity of rice and potato, it would thus be desirable to develop rice or potato with constant and high chlorophyll level. Moreover, children who do not like eating vegetable can obtain chlorophyll from rice or potato.

### SUMMARY OF THE INVENTION

Accordingly, the present invention provides plants with constant and high chlorophyll level. More particularly, the present invention provides transgenic plants, and plant tissues transformed with a specific DNA construct.

In one aspect of the present invention is to provide a DNA construct which includes a specific promoter operably linked to a nucleotide sequence encoding an amino acid sequence comprising specific amino acids, the specific amino acids selected from the group consisting of amino acids of SEQ ID NO:2 (cytosolic Glutamyl-tRNA synthetase), amino acids of SEQ ID NO:4 (organellar Glutamyl-tRNA synthetase), and a combination of the amino acids of SEQ ID NO:2 and SEQ ID NO:4.

In another aspect of the present invention is to provide a transgenic plant which contains the DNA construct including a specific promoter operably linked to a nucleotide sequence encoding an amino acid sequence of cytosolic Glutamyl-tRNA synthetase and a nucleotide sequence encoding an amino acid sequence of organellar Glutamyl-tRNA synthetase.

In yet another aspect of the present invention is to provide a transgenic plant tissue which contains the DNA construct including a specific promoter operably linked to a nucleotide sequence encoding an amino acid sequence of cytosolic Glutamyl-tRNA synthetase and a nucleotide sequence encoding an amino acid sequence of organellar Glutamyl-tRNA synthetase.

Additionally, in another aspect of the present invention is to provide a method of producing a transgenic plant. The method includes: transforming a plant tissue with a DNA construct including a specific promoter operably linked to a nucleotide sequence encoding an amino acid sequence of cytosolic Glutamyl-tRNA synthetase and a nucleotide sequence encoding an amino acid sequence of organellar Glutamyl-tRNA synthetase; generating a whole plant from the transformed plant tissue; optionally multiplying the whole plant; and harvesting tissues from the whole plant or multiplied whole plants.

The aspect of the present invention will no doubt become obvious to those of ordinary skill in the art after reading the following detailed description of the preferred embodiment, which is illustrated in the various figures and drawings.

### BRIEF DESCRIPTION OF THE APPENDED DRAWINGS

FIG. 1A to FIG. 1C are depictions of DNA construct for rice transformation.

FIG. 2A to FIG. 2C are depictions of DNA construct for potato transformation.

### DETAILED DESCRIPTION OF THE INVENTION

The present invention provides a specific DNA construct which includes a specific promoter and two specific genes. In addition, both of these two specific genes can help to increase the level of chlorophyll in plants.

At first, aminoacyl-tRNA synthetases (ARSs) play a critical role in protein synthesis by catalyzing the addition of amino acids to their cognate tRNAs. The specificity of aminoacyl-tRNA synthesis in pairing the appropriate tRNAs and amino acids is a key determinant in faithful transmission of genetic information. Protein synthesis in plants takes place in the cytosol, mitochondria, and chloroplasts, and these compartments do not require full sets of unique ARSs encoded by separate nuclear genes. In general, plant ARSs are classified into two groups based on their substrate specificity: the cytosolic enzymes that most efficiently aminoacylate plant or yeast cytosolic tRNAs, and the organellar enzymes that aminoacylate organelle or Escherichia coli tRNAs.

Other than the role in the protein synthesis, Glutamyl-tRNA synthetase, one of the ARS, is also the first enzyme of the chlorophyll synthesis. Studies have reported that inactivation of organellar glutamyl- and seryl-tRNA synthetases leads to developmental arrest of chloroplasts and mitochondria in higher plants (Kim, Y. K., et al. J. Biol. Chem. 280: 37098-37106 (2005)). Moreover, the content of chlorophyll is also reduced by the nactivation of organellar glutamyl- and seryl-tRNA synthetases. According to the study, it is obvious that the Glutamyl-tRNA synthetase plays a key role in regulation of the content level of chlorophyll and the differentiation of chloroplasts.

Accordingly, the present invention is tended to transfer the cytosolic Glutamyl-tRNA synthetase and organellar Glutamyl-tRNA synthetase into rice and potato.

In a preferred embodiment of the present invention, a DNA construct is provided. The DNA construct includes a specific promoter operably linked to a nucleotide sequence encoding an amino acid sequence consisting of amino acids of SEQ ID NO:2, cytosolic Glutamyl-tRNA synthetase, and/or a nucleotide sequence encoding an amino acid sequence consisting of amino acids of SEQ ID NO:4, organellar Glutamyl-tRNA synthetase.

In an embodiment of the present invention, the construct further includes a second specific promoter, such as a CaMV35S promoter. Moreover, in the embodiment, the construct also includes a marker gene, such as a gene that encoded a phosphomannose isomerase (PMI), operably linked to the second specific promoter. Therefore, the second specific promoter can drive the expression of the marker gene.

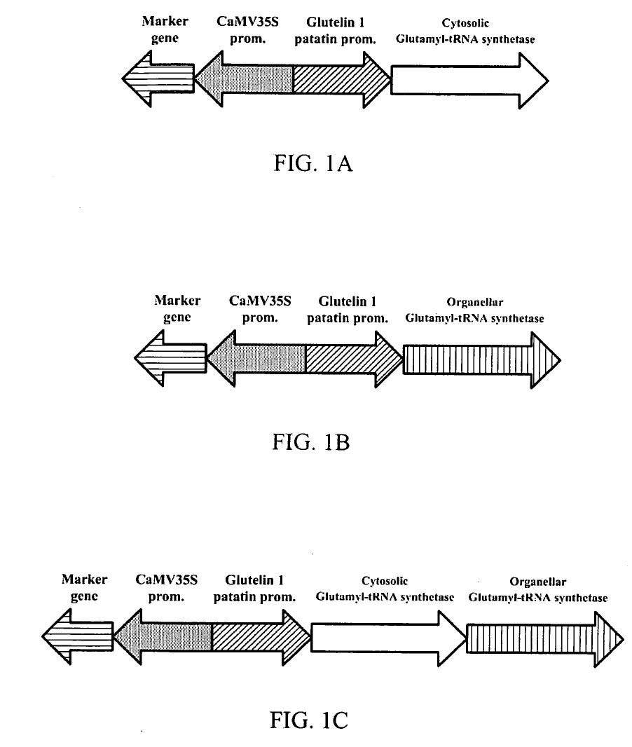

As shown in FIG. 1A, the first specific promoter of the construct in one embodiment of the present invention is a glutelin 1 promoter. Moreover, the DNA construct also includes the nucleotide sequence (SEQ ID NO:1) that encodes the cytosolic Glutamyl-tRNA synthetase driven by the glutelin 1 promoter, the CaMV35S promoter, and the marker gene driven by the CaMV35S promoter.

As shown in FIG. 1B, the first specific promoter of the construct in one embodiment of the present invention is a glutelin 1 promoter. Moreover, the DNA construct also includes the nucleotide sequence (SEQ ID NO:3) that encodes the organellar Glutamyl-tRNA synthetase driven by the glutelin 1 promoter, the CaMV35S promoter, and the marker gene driven by the CaMV35S promoter.

Furthermore, as shown in FIG. 1C, the DNA construct can include both of the nucleotide sequence (SEQ ID NO:1) that encodes the cytosolic Glutamyl-tRNA synthetase and the nucleotide sequence (SEQ ID NO:3) that encodes the organellar Glutamyl-tRNA synthetase.

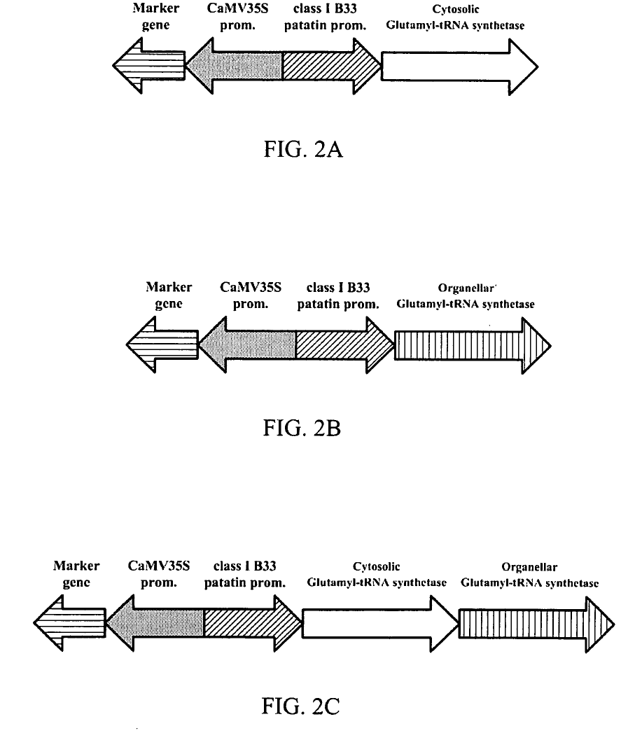

In another embodiment of the present invention, the first specific promoter of the DNA construct is a class I B33 patatin promoter as shown in FIG. 2A to FIG. 2C. It should be noticed that the specific promoters should not be limited to those promoters described above.

In a preferred embodiment of the present invention, a transgenic plant including the DNA construct described above is provided. Additionally, the plant can be a monocot or a dicot. In practice, the plant can be rice, wheat, barley, rye, peanut, corn, maize, potato, sweet potato, beet, radish, onion, garlic, eggplant, pepper, carrot, pumpkin, zucchini, cucumber, apple, pear, melon, citrus, strawberry, grape, raspberry, pineapple, soybean, tomato, sorghum, and sugarcane.

In another preferred embodiment of the present invention, a transgenic plant tissue comprising the DNA construct described above is provided. Furthermore, the plant tissue can be selected from but not limited to fruit, stem, root, seed, and tuber, such as rice seed and potato tuber.

Additionally, a method of producing the transgenic plants and plant tissues is also provided in the present invention. The method includes four major steps as follow:

First of all, transforming a plant tissue with the DNA construct as described above. Then generating a whole plant from the transformed plant tissue. Afterwards, optionally multiplying the whole plant. Finally, harvesting tissues from the whole plant or multiplied whole plants. The detailed description of the method will be disclosed in specific examples below. Please notice that the establishment of DNA constructs used herein is shown in Example 1, the transformation of rice is shown in Example 2, and the transformation of potato is shown in Example 3.

### EXAMPLES

### Example 1

### Establishment of DNA Constructs

A 2760 bp DNA fragment (SEQ ID NO:1) that encoded the cytosolic Glutamyl-tRNA synthetase (SEQ ID NO:2) of Arabidopsis thaliana and a 2055 bp DNA fragment (SEQ ID NO:3) that encoded the organellar Glutamyl-tRNA synthetase (SEQ ID NO:4) of Arabidopsis thaliana were isolated respectively. Please refer to FIG. 1 and FIG. 2 for some examples of DNA constructs established herein. DNA constructs contain each of the DNA fragments or both of the DNA fragments were established respectively. The DNA constructs further contain specific promoter(s), such as CaMV35S promoter, glutelin 1 promoter, and class I B33 patatin promoter. Moreover, the DNA constructs can contain a marker gene, such as a phosphomannose isomerase gene. The DNA constructs were individually delivered into the rice or potato genome via Agrobacterium-mediated transformation as described in Example 2 and Example 3, respectively.

### Example 2

### Rice Transformation

Germination: Sterilize rice seeds with 30% bleach solution, then wash with autoclaved water and spread the sterilized rice seeds onto MS medium (MS+vitamin basal medium, 30 g/L sucrose, 0.7% agar, pH5.8) plate. Grow them in the dark, at 30° C. for 3-5 days.

Induction of Callus: Transfer germinated rice seeds to CIM plates (N6+vitamin basal medium, 0.3 g/L casamino acid, 2.8 g/L raline, 2 mg/L 2,4-D, 100 mg/L myo-inositol, 30 g/L sucrose, 0.7% agar, pH 5.7), and perform callus induction, and then incubate at 30° C. with continuous illumination; subculture them every two weeks.

Infection: Infect subcultured callus cells after 1 week with Agrobacterium suspension cells containing the DNA construct. Agrobacterium suspension cells can be diluted to OD₆₀₀ₙₘ=0.6-0.8 with AAM medium solution (440 mg/L CaCl₂2H₂O, 370 mg/L MgSO₄7H₂O, 170 mg/L KH₂PO₄, 37.5 mg/L Fe-EDTA, 6.2 mg/L H₃BO₃, 22.3 mg/L MnSO₄4H₂O, 8.6 mg/L ZnSO₄7H₂O, 0.83 mg/L KI, 0.25 mg/L Na₂MoO₄2H₂O, 0.025 mg/L CuSO₄5H₂O, 0.02 mg/L CoCl₂6H₂O; vitamins: 1 mg/L thymine HCl, 1 mg/L pyridoxine HCl, 10 mg/L nicotinic acid; amino acids: 75 mg/L glycine, 877 mg/L L-glutamine, 266 mg/L L-aspartic acid, 228 mg/L L-arginine; 68.5 g/L sucrose, 500 mg/L casamino acid, 36 g/L glucose, 100 μM acetosyringone, pH=5.2), and pour them into the plates containing callus cells, then incubate for 15 min.

Co-incubation: Take inoculated callus cells out and remove excess agrobacteria with absorbing paper and then incubate them onto 2N6I-AS medium plate (N6+vitamin basal medium, 1 g/L casamino acid, 2 mg/L 2,4-D, 100 mg/L myo-inositol, 100 μM acetosyringone, 30 g/L sucrose, 10 g/L glucose, 0.7% agar, pH 5.2) at 28° C. for 3 days in the dark.

Screening: After co-incubation, wash callus cells with AA2 medium (440 mg/L CaCl₂2H₂O, 370 mg/L MgSO₄7H₂O, 170 mg/L KH₂PO₄, 37.5 mg/L Fe-EDTA, 6.2 mg/L H₃BO₃, 22.3 mg/L MnSO₄4H₂O, 8.6 mg/L ZnSO₄7H₂O, 0.83 mg/L KI, 0.25 mg/L Na₂MoO₄2H₂O, 0.025 mg/L CuSO₄5H₂O, 0.02 mg/L CoCl₂6H₂O; vitamins: 1 mg/L thymine HCl, 1 mg/L pyridoxine HCl, 10 mg/L nicotinic acid; amino acids: 75 mg/L glycine, 877 mg/L L-glutamine, 266 mg/L L-aspartic acid, 228 mg/L L-arginine; 30 g/L sucrose, 2 mg/L 2,4-D, pH=5.7) for 30 min, repeat 2 more times and remove excess medium solution, then transfer to selection medium plates 2N6I-CH(N6+vitamin basal medium, 1 g/L casamino acid, 2 mg/L 2,4-D, 100 mg/L myo-inositol, 30 g/L sucrose, 0.7% agar, pH 5.2) for screening at 30° C. with continuous light.

Rebirth: Transfer less than 3 mm size of newborn callus tissues to selection medium RM plates (400 mg/L KH₂PO₄, 2830 mg/L KNO₃, 463 mg/L (NH₄)₂SO₄, 185 mg/L MgSO₄7H₂O, 166 mg/L CaCl₂2H₂O, 27.85 mg/L FeSO₄7H₂O, 37.25 mg/L Na-EDTA, 1.8 mg/L ZnSO₄7H₂O, 2.5 mg/L MnSO₄4H₂O, 1.6 mg/L H₃BO₃, 0.83 mg/L KI; N6 vitamin, 0.5 mg/L NAA, 5 mg/L Kinetin, 30 g/L sucrose, 5 g/L glucose, 100 mg/L myo-inositol, 0.7% agar, pH 5.7) and then incubate at 26.5° C. with continuous light.

Induction of rooting: Transfer 3-5 cm shoots to selection medium MS+2,4-D plate, incubate further until plants are 10-15 cm, and then transfer them to soil for further growth.

### Example 3

### Potato Transformation

Sterile seedlings: Sterilize potato tubers with 30% bleach, then wash with autoclaved water, and incubate them onto MS medium plates for germination.

Preculture: Use young leaves of sterile seedlings for preculture. Cut young leaves into segments with 5 mm width, transfer them with upper surface of leaves down to medium HH plates (MS+vitamin basal medium, 3% sucrose, 10 mg/L NAA, 10 mg/L Zeatin riboside, 0.7% agar, pH 5.6-5.8) for preculture and then incubate at 20-22° C. with photoperiod 16 hr light/8 hr dark and light intensity 60 μE/m²s.

Infection: Dilute agrobacterium cells to OD₆₀₀ₙₘ=0.6-0.8 with COD medium (MS+vitamin basal medium, 3% sucrose, 200 μM acetosyringone). Incubate with precultured leave segments for 10 min.

Co-culture: Transfer leaf segments with upper surface down to medium LSR1 plates (containing 200 μM acetosyringone, MS+vitamin basal medium, 3% sucrose, 0.2 mg/L NAA, 2 mg/L Zeatin riboside, 0.02 mg/L GA₃, 0.7% agar, pH 5.6-5.8) for co-culture at 20-22° C. with photoperiod 16 hr light/8 hr dark and light intensity 60 μE/m²s.

Induction of callus: Transfer co-cultured leaf segments to selection medium LSR1T plates (LSR1 medium+200 mg/L timentin) and incubate at 20-22° C. with photoperiod 16 hr light/8 hr dark and light intensity 100 μE/m²s.

Induction of rebirth: Transfer leaf segments with callus containing transgenes onto LSR2 medium plates (MS+vitamin basal medium, 3% sucrose, 2 mg/L Zeatin riboside, 0.02 mg/L GA₃, 0.7% agar, pH 5.6-5.8) for induction of explants, replace with fresh medium every two weeks. Wait until the explants are 1-2 cm and then transfer to CM medium plates (MS+vitamin basal medium, 2% sucrose, 0.7% agar, pH 5.6-5.8), also can include ethylene inhibitor (1 mg/L silver thiosulphate) in CM plates for inhibition of overgrowth of shoots.

Induction of rooting: Transfer shoots onto RT medium plates (MS+vitamin basal medium, 2.5% sucrose, 0.2 mg/L indoleacetic acid, 0.7% agar, pH 5.6-5.8) for induction of rooting.

All of the features disclosed in this specification may be combined in any combination. Each feature disclosed in this specification may be replace by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Accordingly, other embodiments are also within the scope of the following claims.

What is claimed is:

1. A DNA construct comprising a first specific promoter operably linked to a nucleotide sequence encoding an amino acid sequence comprising specific amino acids, the specific amino acids selected from the group consisting of amino acids of SEQ ID NO:2, amino acids of SEQ ID NO:4, and a combination of the amino acids of SEQ ID NO:2 and SEQ ID NO:4.

2. The DNA construct according to claim 1, further comprising a second specific promoter.

3. The DNA construct according to claim 2, wherein the second specific promoter is a CaMV35S promoter.

4. The DNA construct according to claim 3, further comprising a marker gene operably linked to the second specific promoter.

5. The DNA construct according to claim 4, wherein the marker gene is a gene that encoded a phosphomannose isomerase (PMI).

6. The DNA construct according to claim 1, wherein the first specific promoter is a glutelin 1 promoter.

7. The DNA construct according to claim 1, wherein the first specific promoter is a class I B33 patatin promoter.

8. A transgenic plant comprising a DNA construct which comprises a specific promoter operably linked to a nucleotide sequence encoding an amino acid sequence comprising specific amino acids, the specific amino acids selected from the group consisting of amino acids of SEQ ID NO:2, amino acids of SEQ ID NO:4, and a combination of the amino acids of SEQ ID NO:2 and SEQ ID NO:4.

9. The transgenic plant according to claim 8, wherein the plant is one selected from the group consisting of rice, wheat, barley, rye, peanut, corn, maize, potato, sweet potato, beet, radish, onion, garlic, eggplant, pepper, carrot, pumpkin, zucchini, cucumber, apple, pear, melon, citrus, strawberry, grape, raspberry, pineapple, soybean, tomato, sorghum, and sugarcane.

10. The transgenic plant according to claim 9, wherein the plant is rice.

11. The transgenic plant according to claim 9, wherein the plant is wheat.

12. The transgenic plant according to claim 9, wherein the plant is carrot.

13. The transgenic plant according to claim 9, wherein the plant is potato.

14. A transgenic plant tissue comprising a DNA construct which comprises a specific promoter operably linked to a nucleotide sequence encoding an amino acid sequence comprising specific amino acids, the specific amino acids selected from the group consisting of amino acids of SEQ ID NO:2, amino acids of SEQ ID NO:4, and a combination of the amino acids of SEQ ID NO:2 and SEQ ID NO:4.

15. The transgenic plant tissue according to claim 14 which is selected from the group consisting of fruit, stem, root, seed, and tuber.

16. The transgenic plant tissue according to claim 15, wherein the seed is a rice seed.

17. The transgenic plant tissue according to claim 15, wherein the tuber is a potato tuber.

18. A method of producing a transgenic plant, comprising:
transforming a plant tissue with a DNA construct comprising a specific promoter operably linked to a nucleotide sequence encoding an amino acid sequence comprising specific amino acids, the specific amino acids selected from the group consisting of amino acids of SEQ ID NO:2, amino acids of SEQ ID NO:4, and a combination of the amino acids of SEQ ID NO:2 and SEQ ID NO:4;
generating a whole plant from the transformed plant tissue;
optionally multiplying the whole plant; and
harvesting tissues from the whole plant or multiplied whole plants.

19. The plant tissue according to claim 18 which is selected from the group consisting of fruit, stem, root, seed, and tuber.

20. The plant according to claim 18, wherein the plant is one selected from the group consisting of rice, wheat, barley, rye, peanut, corn, maize, potato, sweet potato, beet, radish, onion, garlic, eggplant, pepper, carrot, pumpkin, zucchini, cucumber, apple, pear, melon, citrus, strawberry, grape, raspberry, pineapple, soybean, tomato, sorghum, and sugarcane.


SEQUENCE LISTING (4 sequences)

SEQ ID NO: 1 - DNA, 2760 bp, Arabidopsis thaliana
agggttctgtttctcgtctctctctcaaacccacattgcacaatccatttctcgtttctctgatttagatccaaagatggatgggatgaagctttcgttcccaccggaaagtccaccactttcagtcatcgttgctctttctctctcagcttctccggtgacgattgattcttccgccgctgcaacaaccgtcccttcttttgtcttctccgacgggaggaaattgaatggagccaccgttcttcttcgctatgttggtcgatcagcgaaaaagcttcctgatttctatggcaacaatgcttttgattcttctcagattgatgagtgggtagattacgcatctgtcttctcttctggttcagagtttgagaatgcttgtggtcgtgttgataagtatctcgagagtagcacgtttcttgttggccattctctttccattgctgatgtcgctatttggtcagctcttgctggaactggtcaaagatgggaaagtttgaggaaatctaaaaagtatcagagtcttgttagatggttcaattcgatattagacgagtacagtgaggtgcttaacaaggttctagcaacttatgttaagaaaggatcagggaagcctgttgctgcacctaagtctaaagatagccaacaagctgtgaaaggagatggtcaggataaaggtaagcctgaagtggacttgccggaagcggagattggaaaggttaaactccggtttgctccagagccaagtggttatcttcacataggacatgctaaggctgcgttgctgaacaagtatttcgctgagcgttaccaaggggaagtgattgtgcgttttgatgatactaaccctgctaaagaaagcaatgagtttgtggataatcttgtgaaggatattgggaccttggggatcaagtatgagaaagtgacatacacttcggactattttcctgaattgatggatatggcggaaaaactgatgcgtgagggtaaggcatatgttgatgacacaccgagggagcagatgcagaaagagaggatggatgggattgattcgaaatgtaggaatcatagcgtcgaggagaatttgaagctatggaaggaaatgattgcaggaagtgagagaggattacagtgctgtgttcgtgggaaattcaacatgcaagatcccaacaaagccatgcgtgacccggtttattaccgatgcaatcctatgtctcaccaccgtatcggggataagtataagatatatccaacatatgactttgcttgcccgtttgttgattcccttgaaggtataacgcatgctcttcggtctagtgagtatcatgaccgaaatgctcagtactttaaagttctggaggatatgggactgcgacaggttcagctttacgaattcagccggttaaacctagtttttacacttctcagtaagcgcaagcttctctggtttgtccaaactggattggttgacgggtgggatgatccacgtttcccgacagtccaaggaattgttcgtagaggtttgaaaatcgaggctctgattcaattcattctcgagcagggggcttcgaagaatctaaatttgatggaatgggacaaactttggtctataaataagagaataattgatcctgtgtgccctagacacactgctgtggttgcagaacgtcgtgtactatttaccttaacggatggtcctgatgagccgtttgttcgcatgataccaaagcacaagaaattcgaaggtgctggagaaaaggcgaccactttcactaagagcatttggctcgaggaagctgatgcgagtgccatatccgttggtgaggaagtaactttgatggattggggaaatgctatcgtaaaggaaatcacaaaggacgaggagggtcgtgtcactgccttatctggtgtcttgaatctccaaggttctgtaaagactacaaagctgaagctgacatggcttcctgatactaatgaattggtcaatctcacattaacagagtttgattatctaatcaccaagaagaagctggaagatgatgatgaagttgctgattttgtgaatcctaacacaaagaaggaaacattggcacttggtgattcgaatatgaggaatctgaaatgtggagatgtgattcagcttgagaggaaaggctatttcagatgtgatgtgccttttgtcaaatcttcaaagcccattgtcttattctccattccagatggaagagccgctaagtgatcaatgaactatttttgagtttcccattttatgaacattgtgttgagaaatttgctctttaggttttaaaacaatgaaaacaaagctttttatcagttctttagtttcatctcataatagttttaaagttgcaaactttatattgattcattcttctttgcttagtgtttacaatataaacaaaaacaaactcatttcttgggtgcgtatctcagtttgatcttcttaacctgtttgtgtttgagctggaatgacttggccagaatgtcatcgggaagagctccaaagagagaggcgacaagtgattgtgttccgggcaattggctatcgaaggctgcgagtacagatgctggactgttggcaatgttcttctggaaatggatgagtcccttggggaaggtgaaaacgtctcccttgttaaggttcttggcgatgagttttccagcggttgttacaaagccaacatagagctgaccttcaaggacgaaaatgatctcggaggcacgtgggtggacatggggtgg

SEQ ID NO: 2 - protein, 719 aa, Arabidopsis thaliana
MDGMKLSFPPESPPLSVIVALSLSASPVTIDSSAAATTVPSFVFSDGRKLNGATVLLRYVGRSAKKLPDFYGNNAFDSSQIDEWVDYASVFSSGSEFENACGRVDKYLESSTFLVGHSLSIADVAIWSALAGTGQRWESLRKSKKYQSLVRWFNSILDEYSEVLNKVLATYVKKGSGKPVAAPKSKDSQQAVKGDGQDKGKPEVDLPEAEIGKVKLRFAPEPSGYLHIGHAKAALLNKYFAERYQGEVIVRFDDTNPAKESNEFVDNLVKDIGTLGIKYEKVTYTSDYFPELMDMAEKLMREGKAYVDDTPREQMQKERMDGIDSKCRNHSVEENLKLWKEMIAGSERGLQCCVRGKFNMQDPNKAMRDPVYYRCNPMSHHRIGDKYKIYPTYDFACPFVDSLEGITHALRSSEYHDRNAQYFKVLEDMGLRQVQLYEFSRLNLVFTLLSKRKLLWFVQTGLVDGWDDPRFPTVQGIVRRGLKIEALIQFILEQGASKNLNLMEWDKLWSINKRIIDPVCPRHTAVVAERRVLFTLTDGPDEPFVRMIPKHKKFEGAGEKATTFTKSIWLEEADASAISVGEEVTLMDWGNAIVKEITKDEEGRVTALSGVLNLQGSVKTTKLKLTWLPDTNELVNLTLTEFDYLITKKKLEDDDEVADFVNPNTKKETLALGDSNMRNLKCGDVIQLERKGYFRCDVPFVKSSKPIVLFSIPDGRAAK

SEQ ID NO: 3 - DNA, 2055 bp, Arabidopsis thaliana
agttatgaggctttaatcgaatttgttgctttgctttgcgactttcatctttccgattaatcaaatccttttatcctctgaaaaatctcaactttcgaagaaaacgaaaatggcgagccttgtctacgggacgccgtggcttagggttagatctttaccggagcttgctccagcttttctcagacggcgtcaatcttctttattttactgttctcggcgaagcttcgcggtggttgcgtgttccactccagtcaacaatggtgggtctgtcagagttcgtttcgcgccttctcctactggtaatctccatgtaggtggagcaaggactgctcttttcaactacttgttcgcgaggtctaaaggagggaaatttgtgctgagaattgaagatacagatttggagagatcaacacgtgaatctgaagcagctgttcttcaggatctctcatggcttggtcttgattgggatgaaggtcctggggttagtggagactttggtccttatcggcaatctgaaagaaatgctttgtataaacaatatgcagagaagcttttagagtcaggtcatgtctatcgatgcttttgctcaagtgaggaacttgtaaagatgaaggagaatgcaaagttgaaacagttgcctccagtatataccgggaaatgggcaacagcttctgatgctgaaatagagcaagagttagaaaagggaacaccttttacttaccggtttcgtgtgccaaaggaaggctctttgaaaattaatgacctgattcgtggcgaggtctgttggaacttggatactcttggggattttgtggtaatgagaagtaatggccaacccgtttacaacttttgtgttacggtagatgatgctaccatggctatatcacatgttataagggctgaagaacacttgcctaatactttgaggcaggctctaatttacaaggctcttaagttcccgatgcctcaatttgcacatgtttctttaattttagctccggatagaagtaaattatcaaagcgacatggggcaacttctgtaggccagtacagagagatgggatatctacctcagggaatggttaactatttggcactcttaggttggggagacgggactgaaaatgaattcttcacactcgaggatcttgttgaaaaattctcgattgaacgtgtcaacaaaagtggcgcgatttttgattcaacaaagttaagatggatgaatggtcaacatctgagggcacttccaaatgagaaactaacaaaacttgttggtgagcgatggaagagcgctggtatcttaacagaatccgaggggagttttgtaaatgaagctgtcgagcttctcaaggatgggattgagttggtgacagattcagacaaagtacttttgaacttgctttcatatcctctacacgctacattggctagccctgaagctaagcctgctgtggaagacaaacttcatgaagtagcagccagcctcatagctgcttatgacagcggagagattccaagcgctttagaagaaggacaaggtgcttggcagaaatgggtgaaagcctttggcaaatccttgaaacgcaaaggtaaatcacttttcatgccactacgagtgttgttaacgggaaaactccatggtcctgagatgggcaccagtattgttctgatttacaaagctggaagtcctggtatagtggttcctcaagctgggtttgtgtccatggaggaacggtttaagattcttagggagatagactgggaagctttgaacaaagatgagagtgtgcctcttgaatctacagccacagtatcaacctgagaaaactcccctttttttttttttttttttttccttcaatatttggttagttggtgagagaaagacagcccagagattttgattcctcgtgcatttcttgtttcccgaaacaaatggcacaaagaaatttgtagattataatgtatcagcaaagcaaacttaggaacaaaccagagagcttgtaacattatcaggccagttttaatttgctttcaatgctctgttttcttaac

SEQ ID NO: 4 - protein, 570 aa, Arabidopsis thaliana
MASLVYGTPWLRVRSLPELAPAFLRRRQSSLFYCSRRSFAVVACSTPVNNGGSVRVRFAPSPTGNLHVGGARTALFNYLFARSKGGKFVLRIEDTDLERSTRESEAAVLQDLSWLGLDWDEGPGVSGDFGPYRQSERNALYKQYAEKLLESGHVYRCFCSSEELVKMKENAKLKQLPPVYTGKWATASDAEIEQELEKGTPFTYRFRVPKEGSLKINDLIRGEVCWNLDTLGDFVVMRSNGQPVYNFCVTVDDATMAISHVIRAEEHLPNTLRQALIYKALKFPMPQFAHVSLILAPDRSKLSKRHGATSVGQYREMGYLPQGMVNYLALLGWGDGTENEFFTLEDLVEKFSIERVNKSGAIFDSTKLRWMNGQHLRALPNEKLTKLVGERWKSAGILTESEGSFVNEAVELLKDGIELVTDSDKVLLNLLSYPLHATLASPEAKPAVEDKLHEVAASLIAAYDSGEIPSALEEGQGAWQKWVKAFGKSLKRKGKSLFMPLRVLLTGKLHGPEMGTSIVLIYKAGSPGIVVPQAGFVSMEERFKILREIDWEALNKDESVPLESTATVST